(19) United States
(12) Patent Application Publication
(10) Pub. No.: US 2009/0227633 A1
(43) Pub. Date: Sep. 10, 2009

(54) Methods to inhibit tumor cell growth by using proton pump inhibitors

(76) Inventor: Bassam Damaj, San Diego, CA (US)

Correspondence Address: TRASKBRITT, P.C., P.O. BOX 2550, SALT LAKE CITY, UT, 84110, US

(21) Appl. No.: 12/074,644

(22) Filed: Mar. 4, 2008

(51) Int. Cl. A61K 31/435; A61P 35/00

(52) U.S. Cl. 514/338

(57) ABSTRACT

Methods of treating one or more growth deregulated cells are disclosed. An effective amount of a pharmaceutical composition including a proton pump inhibitor is administered thereby treating a growth deregulated cell outside of the gastric lumen of a subject.

### TECHNICAL FIELD

Embodiments of the invention relate, in part, to methods of treating tumor cells by administration of a proton pump inhibitor.

### BACKGROUND

Cytotoxic agents remain the mainstay of cancer treatment due to high unmet needs in the disease. Because the oral and gastrointestinal mucosa is often significantly damaged by cancer therapy, management of these problems is an important challenge for oncologists. Such treatment complications are generally not severe or life threatening, but they can result in both treatment delays and dose reductions in potentially curative regimens. Numerous therapeutic approaches have been evaluated as prophylaxis or treatment for mucosal damage in patients undergoing cancer therapy. The results of large-scale, placebo-controlled, comparative trials demonstrate that administration of a proton pump inhibitor can provide both significant symptom relief and prophylaxis against upper gastrointestinal ulceration in patients receiving cancer chemotherapy.

In addition to combating chemotherapy side effects, proton pump inhibitors have been used to lower a tumor cell's resistance to cytotoxic agents. The mechanisms underlying this phenomenon appear to take advantage of functions involved in the control of cell homeostasis. One mechanism of resistance may be alteration of the tumor microenvironment via changes in the pH gradient between the extracellular environment and the cell cytoplasm and/or in the pH gradient between the cell cytoplasm and lysosomal compartments. The extracellular (i.e., interstitial) pH of solid tumors is substantially more acidic than that of normal tissues, and the acidic pH of the tumor microenvironment may impair the uptake of weakly basic chemotherapeutic drugs.

Cancer is worldwide health problem. There is an on-going need for new methods to treat and/or limit tumor growth that augments or replaces currently used methods, compounds and compositions.

### BRIEF SUMMARY

Described are methods of treating tumor cells by administering a pharmaceutically acceptable composition that may include a proton pump inhibitor to tumor cells. The proton pump inhibitor or a pharmaceutically acceptable salt thereof may be administered as a pharmaceutically acceptable composition and may decrease tumor cell volume. The pharmaceutically acceptable composition may include a buffering agent.

The pharmaceutically acceptable composition may include about 20 mg to about 400 mg of lansoprazole. After administration, the pharmaceutically acceptable composition may interact with tumor cells outside of the gastric lumen. The proton pump inhibitor may induce apoptosis in the tumor cells and/or may lower the pH of the tumor cells. In an embodiment, the proton pump inhibitor may induce apoptosis in the cancer cells by modifying the K⁺ level of the tumor cells.

In an embodiment, the proton pump inhibitor may be a substituted benzimidazole compound having H⁺/K⁺ ATPase inhibiting activity. The proton pump inhibitor is selected from the group consisting of lansoprazole, omeprazole, rabeprazole, esomeprazole, pantoprazole, pariprazole, leminoprazole, SCH 28080, and enantiomers, isomers, free bases, salts, and mixtures thereof. In an embodiment, a composition may be administered in a dosage of about 180 mg/day of lansoprazole. The proton pump inhibitor may be administered in an amount of about 10 mg/kg to about 100 mg/kg.

Tumor cells may be selected from a group consisting of: RPM18226, NC37, MC/CAR, SUDHM, RPMI6666, GDM-1, MOLT3, J45-01, MCF7, HL60 clone 15, P116, SW620, MV-4-11, SKMEL5, DAUDI, DOHH2, HUT102, CCRF-CEM, HUT78, A3, MDA-MB-435, MDA-MB-231, and RS4.11.

In one embodiment, the tumor cells are selected from a group consisting of: ES-2, IGROV 1, OVCAR5, OVCAR8, J-gamma-1, KU812, NK92MI, 786-O, A498, H522, SNB19, OVCAR4, H9, HH, EKVX, OVCAR5, UACC257, H226, UO-31, NAMALWA, SKMEL28, SKMEL2, M14, H322M, HCC2998, HL60, HT29, A549, RXF393, PC3, H460, MC116, MOLT4, JMI, HOP-62, HCT-15, SF-539, SF295, ST486, U251, and UACC-62. The tumor cells may be associated with a disease selected from the group consisting of: carcinoma, lymphoma, blastoma, myeloma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, and hepatic carcinoma.

A second chemotherapeutic agent may be administered with a proton pump inhibitor. In an embodiment, a proton pump inhibitor, such as lansoprazole, and a second chemotherapeutic agent are administered together in the same or separate dosage form. The proton pump inhibitor, such as lansoprazole, and a second chemotherapeutic agent may be administered before, after or simultaneously.

Also described herein are methods of treating one or more growth deregulated cells by administering to a subject an effective amount of a pharmaceutical composition. In an embodiment, the pharmaceutical composition may include lanzoprazole, for example, in a dose of about 120 mg to about 400 mg or more per day. In one embodiment, the lanzoprazole is administered in a dose of about 120 mg to about 300 mg, wherein upon administration to the subject the composition interacts with a mass of growth deregulated cells outside of the subject's gastric lumen. The lansoprazole may induce apoptosis in the growth deregulated cells and the mass of growth deregulated cells may be reduced in size after about three weeks from the administration.

Also described are methods of treating lymphoma including administering to a patient in need thereof a pharmaceutically effective amount of lansoprazole or a pharmaceutically acceptable salt thereof.

Also described are methods of treating a patient having a cancerous tumor. The method may include administering to a patient in need thereof lansoprazole or pharmaceutically acceptable salts thereof in an amount effective to inhibit the growth of the tumor. The inhibition of growth may be measured as a delay in tumor doubling time. The tumor doubling time may be extended by a factor of at least two. The volume of the tumor may be reduced by at least 10%.

Also described are methods of treating cancer. The methods may include administering to a patient in need thereof a pharmaceutically effective amount of lansoprazole or a pharmaceutically acceptable salt thereof. The cancer may be selected from the group consisting of: carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, and hepatic carcinoma.

Also described as methods of treating leukemia. The methods may include administering to a patient in need thereof a pharmaceutically effective amount of lansoprazole or a pharmaceutically acceptable salt thereof.

Also described are methods of treating kidney cancer. The methods may include administering to a patient in need thereof a pharmaceutically effective amount of lansoprazole or a pharmaceutically acceptable salt thereof.

In embodiments of the invention, lansoprazole may be administered at a dosage of about 120 mg/day to about 300 mg/day. Lansoprazole may be administered at a dosage of about 10 mg/kg/day to about 150 mg/kg/day. The survival rate of a subject receiving a pharmaceutical composition of the invention may be greater than about 15% as compared to a patient administered a placebo.

### BRIEF DESCRIPTION OF THE SEVERAL VIEWS OF THE DRAWINGS

FIGS. 1A-1AU depict experimental results of tumor cells from the following cell lines treated with lansoprazole: 786-O (FIG. 1A), ACHN (FIG. 1B), A498 (FIG. 1C), H226 (FIG. 1D), H522 (FIG. 1E), HOP92 (FIG. 1F), SNB19 (FIG. 1G), SUDHL4 (FIG. 1H), OVCAR4 (FIG. 1I), IGROV1 (FIG. 1J), H9 (FIG. 1K), UO-31 (FIG. 1L), HH (FIG. 1M), DAUDI (FIG. 1N), LOXIMVI (FIG. 1O), NAMALWA (FIG. 1P), EKVX (FIG. 1Q), DOHH2 (FIG. 1R), SNB75 (FIG. IS), SKMEL28 (FIG. 1T), SKMEL5 (FIG. 1U), SKMEL2 (FIG. 1V), OVCAR5 (FIG. 1W), M14 (FIG. 1X), UACC257 (FIG. 1Y), H322M (FIG. 1Z), KM12 (FIG. 1AA), H332M (FIG. 1AB), HUT102 (FIG. 1AC), HL60 (FIG. 1AD), CCRF-CEM (FIG. 1AE), HUT78 (FIG. 1AF), HT29 (FIG. 1AG), HCT116 (FIG. 1AH), CAKI-1 (FIG. 1AI), DU-145 (FIG. 1AJ), A549 (FIG. 1AK), H460 (FIG. 1AL), A3 (FIG. 1AM), MDA-MB-231 (FIG. 1AN), MALME-3M (FIG. 1AO), J-gamma-1 (FIG. 1AP), RXF393 (FIG. 1AQ), RS4.11 (FIG. 1AR), PC3 (FIG. 1AS), OVCAR3 (FIG. 1AT), and MDA-MB-435 (FIG. 1AU).

FIGS. 2A-2O depict experimental results of tumor cells from the following cell lines treated with lansoprazole: Du-145 (FIG. 2A), J45-01 (FIG. 2B), MOLT3 (FIG. 2C), HUT78 (FIG. 2D), J-gamma-1 (FIG. 2E), MCF7 (FIG. 2F), Colo205 (FIG. 2G), HL60 clone 15 (FIG. 2H), T47D (FIG. 2I), P116 (FIG. 2J), Ku812 (FIG. 2K), SW620 (FIG. 2L), NK92MI (FIG. 2M), MV-4-11 (FIG. 2N), and HS578T (FIG. 20).

FIGS. 3A-3K depict experimental results of tumor cells from the following cell lines were treated with lansoprazole, omeprazole, and SCH 28080: IGROV1 (FIG. 3A), OVCAR8 (FIG. 3B), ES-2 (FIG. 3C), CAOV3 (FIG. 3D), OVCAR5 (FIG. 3E), SUGHL4 (FIG. 3F), RPMI8226 (FIG. 3G), RPMI6666 (FIG. 3H), NC37 (FIG. 3I), GDM-1 (FIG. 3J), and MC/CAR (FIG. 3K).

FIGS. 4A-4C depict mean tumor volume over time in a nude mouse model treated with lansoprazole. The nude mice were exposed to tumor cell lines G401 (FIG. 4A), HEPG2 (FIG. 4B), and JR (FIG. 4C).

FIGS. 5A-5C depict mean tumor volume over time in a nude mouse model treated with lansoprazole. (The nude mice were exposed to tumor cell lines G401 (FIG. 5A), RS1184 B (FIG. 5B), and SR (FIG. 5C).

FIGS. 6A-6M show depicts results of contacting various tumor cells with lansoprazole: MC116 (FIG. 6A), HOP-62 (FIG. 6B), JMI (FIG. 6C), RPMI6666 (FIG. 6D), MOLT4 (FIG. 6E), HCT-15 (FIG. 6F), RS1184 (FIG. 6G), ST486 (FIG. 6H), U251 (FIG. 6I), SF-539 (FIG. 6J), SF295 (FIG. 6K), UACC-62 (FIG. 6L), and SR (FIG. 6M).

### DETAILED DESCRIPTION OF THE INVENTION

In general, the present disclosure relates to methods of treating a tumor by administering a pharmaceutical composition including a proton pump inhibitor. Additionally, the present disclosure relates to methods of treating a tumor by contacting or administering a pharmaceutical composition including a proton pump inhibitor in the presence or absence of a cytotoxic drug or chemotherapeutic agent with or to a tumor cell. While the present disclosure may be embodied in many different forms, several specific embodiments are discussed herein with the understanding that the present disclosure is to be considered only as an exemplification of the principles of the invention, and it is not intended to limit the invention to the embodiments illustrated.

“Treating” or “treatment” as used herein does not require a complete cure. It means that the symptoms of the underlying disease or condition are at least reduced, and/or that one or more of the underlying cellular, physiological, or biochemical causes or mechanisms causing the symptoms are reduced and/or eliminated. It is understood that reduced, as used in this context, means relative to the state of the disease or condition, including the molecular state of the disease or condition, not just the physiological state of the disease or condition.

As used herein, the term “tumor” represents a single cell or multiple cells. “Tumor” as used herein refers to any growth deregulated cell which may be part of a mass of tissue.

As used herein, the term “growth deregulated cell” represents a single or multiple cancerous cells characterized by unregulated cell growth. A growth deregulated cell may form a mass of tissue that results from excessive cell growth or proliferation, either benign (noncancerous) or malignant (cancerous) including pre-cancerous lesions.

As used herein, the terms “cancer” and “cancerous” refer to or describe the physiological condition in mammals in which a population of cells are characterized by unregulated cell growth. Examples of such may include, but are not limited to, carcinoma, lymphoma, blastoma, sarcoma, and leukemia. More particular examples of such cancers include, but are not limited to, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

As used herein, the term “proton pump inhibitor” (PPD) shall mean any substituted benzimidazole possessing pharmacological activity as an inhibitor of H⁺, K⁺-ATPase, including, but not limited to, omeprazole, lansoprazole, pantoprazole, rabeprazole, dontoprazole, perprazole (s-omeprazole magnesium), habeprazole, ransoprazole, pariprazole, and leminoprazole in neutral form or a salt form, a single enantiomer or isomer or other derivative or an alkaline salt of an enantiomer of the same. Proton pump inhibitors are also functionally defined as compounds that act by irreversibly blocking the hydrogen/potassium adenosine triphosphatase enzyme system (the H⁺/K⁺ ATPase, or more commonly just gastric proton pump) of the gastric parietal cell. The proton pump is the terminal stage in gastric acid secretion, being directly responsible for secreting H⁺ ions into the gastric lumen, making it an ideal target for inhibiting acid secretion. Proton pump inhibitors also include the compound SCH 28080.

“Derivatives and analogs of lansoprazole” include, as disclosed in U.S. Pat. No. 4,628,098, the complete disclosure of which is hereby incorporated by reference, compounds having the general formula (I) below and stereoisomers and pharmaceutically acceptable salts thereof:

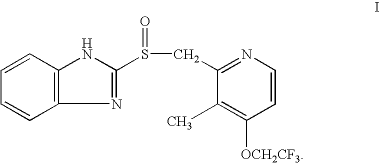

“Pharmaceutically acceptable salts of lansoprazole” refers to those salts of lansoprazole derivatives that retain the biological effectiveness and properties of the free acids or free bases and that are not otherwise unacceptable for pharmaceutical use. Pharmaceutically acceptable salts of lansoprazole derivatives include salts of acidic or basic groups which may be present in the lansoprazole derivatives. Derivatives of lansoprazole that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as chloride, bromide, iodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Derivatives of lansoprazole that include an amino moiety can also form pharmaceutically acceptable salts with various amino acids, in addition to the acids mentioned above. Derivatives of lansoprazole that are acidic in nature are capable of forming a wide variety of salts with various inorganic and organic bases. Suitable base salts are formed from bases that donate cations to form non-toxic salts, suitable cations include, but are not limited to, sodium, aluminum, calcium, lithium, magnesium, potassium, zinc and diethanolamine salts. For a review on pharmaceutically acceptable salts see Berge et al., J. Pharm. Sci., 66, 1-19 (1977), and REMINGTON'S PHARMACEUTICAL SCIENCES, 18th Ed. (1990, Mack Publishing Co., Easton, Pa.), which are incorporated herein by reference.

The phrase, “therapeutically effective” refers to the ability of an active ingredient, for example, lansoprazole, to elicit the biological or medical response that is being sought by a researcher, veterinarian, medical doctor or other clinician. Non-limiting examples include, but are not limited to, reduction of tumor size in a patient, extended survival time, and the like.

The phrase, “therapeutically effective amount” includes the amount of an active ingredient, for example, lansoprazole, that will elicit the biological or medical response that is being sought by the researcher, veterinarian, medical doctor or other clinician. The compounds of the invention may be administered in amounts effective at reducing tumor size and/or extending survival time. Alternatively, a therapeutically effective amount of an active ingredient is the quantity of the compound required to achieve a desired therapeutic and/or prophylactic effect, such as the amount of the active ingredient that results in the prevention of or a decrease in the symptoms associated with the condition (for example, to meet an end-point). An effective amount may include an amount with or without undue adverse side effects, including but not limited to, raising of gastric pH, reduced gastrointestinal bleeding, reduction in the need for blood transfusion, improved survival rate, more rapid recovery, parietal cell activation and H⁺, K⁺-ATPase inhibition or improvement or elimination of symptoms, and other indicators as are selected as appropriate measures by those skilled in the art.

The terms, “pharmaceutically acceptable” or “pharmacologically acceptable” refer to molecular entities and compositions that do not produce an adverse, allergic or other untoward reaction when administered to an animal, or to a human, as appropriate. The term, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Any suitable route of administration may be employed for providing a subject with an effective amount of lansoprazole. Rectal, parenteral (non-limiting examples include subcutaneous, intramuscular, intravenous), transdermal, topical, oral administration, ocular, otic, nasal administration and like forms of administration are possible. Oral dosage forms include tablets, troches, dispersions, suspensions, solutions, capsules, and the like.

The composition may comprise dry formulations, solutions and/or suspensions of the proton pump inhibitors. As used herein, the terms “suspension” and “solution” are interchangeable with each other and mean solutions and/or suspensions of the substituted benzimidazoles.

In certain embodiments, after administration of the PPI, the drug is absorbed through the gastric lumen and delivered via the bloodstream to various tissues and cells of a subject including various tumor cells. Without being bound by any particular theory, it is believed that the PPI comes in contact with tumor cells and prevents the tumor cell from expunging H⁺ by inhibiting the HIK ATPase proton pump. Inhibition of the proton pump may raise the intracellular pH of the tumor cells, eventually leading to cell apoptosis.

A pharmaceutical composition including a proton pump inhibitor such as lansoprazole, omeprazole, or other proton pump inhibitor and derivatives thereof may be used for the treatment or prevention of cancer. For example, a pharmaceutical composition comprising a proton pump inhibitor such as lansoprazole may be used for the treatment or prevention of ovarian cancer, lymphoma, B lymphocyte myeloma, Hodgkins lymphoma, breast cancer, leukocyte cancer, liver cancer, ovarian cancer, bladder cancer, prostate cancer, skin cancer, bone cancer, brain cancer, leukemia cancer, lung cancer, colon cancer, CNS cancer, melanoma cancer, renal cancer or cervical cancer. Treatment of these conditions may be accomplished by administering to a subject an effective amount of the pharmaceutical composition according to embodiments of the present invention.

The dosage range of lansoprazole or other proton pump inhibitors such as substituted benzimidazoles and derivatives thereof may range from approximately <20 mg/day to approximately 400 mg/day or more. The approximate daily oral dosage may be about 20 mg of esomeprazole, about 360 mg omeprazole, about 240 mg pantoprazole, about 120 mg rabeprazole, and the pharmacologically equivalent doses of other PPIs including, but not limited to habeprazole, pariprazole, dontoprazole, ransoprazole, perprazole (s-omeprazole magnesium), and leminoprazole. Alternatively, the dosage range of lansoprazole or other proton pump inhibitors such as substituted benzimidazoles and derivatives thereof may range from approximately <10 mg/kg to approximately 100 mg/kg or more.

A pharmaceutical composition of the proton pump inhibitors utilized in embodiments of the present invention may be administered in any form to a subject. This may be accomplished, for example, by administering the solution via a nasogastric (ng) tube or other indwelling tubes placed in the GI tract.

The terms, “individual,” “patient,” or “subject” are used interchangeably herein and include any mammal, including animals, for example, primates, for example, humans, and other animals, for example, dogs, cats, swine, cattle, sheep, and horses. The compounds of the invention can be administered to a mammal, such as a human, but can also be other mammals, for example, an animal in need of veterinary treatment, for example, domestic animals (for example, dogs, cats, and the like), farm animals (for example, cows, sheep, pigs, horses, and the like) and laboratory animals (for example, rats, mice, guinea pigs, and the like). As used herein, the term “subject” includes cells in a culture or tumor tissue cells.

In one embodiment, a liquid oral pharmaceutical composition may be prepared by mixing lansoprazole (PREVACID®) and/or other proton pump inhibitor or derivatives thereof with a solution including a buffering agent. For example, lansoprazole and/or another proton pump inhibitor(s), may be mixed with a sodium bicarbonate solution to achieve a desired final lansoprazole (or other PPI) concentration. As an example, the concentration of lansoprazole in the solution may range from approximately <180 mg/ml to approximately 300.0 mg/ml or more.

In one embodiment, a liquid oral pharmaceutical composition may be prepared by mixing lansoprazole (PREVACID®) and/or other proton pump inhibitor or derivatives thereof with a solution including a buffering agent. For example, lansoprazole and/or another proton pump inhibitor(s), may be mixed with a sodium bicarbonate solution to achieve a desired final lansoprazole (or other PPI) concentration. As an example, the concentration of lansoprazole in the solution may range from approximately <180 mg/ml to approximately 300.0 mg/ml or more.

Although sodium bicarbonate is one buffering agent which is employed in certain embodiments of the invention to protect the PPI against acid degradation, many other weak and strong bases (and mixtures thereof) may be utilized. For the purposes of this application, “buffering agent” shall mean any pharmaceutically appropriate weak base or strong base (and mixtures thereof) that, when formulated or delivered with (e.g., before, during and/or after) the PPI, functions to substantially prevent or inhibit the acid degradation of the PPI by gastric acid sufficient to preserve the bioavailability of the PPI administered. The buffering agent, if used, may be administered in an amount sufficient to substantially achieve the above functionality. Moreover, the buffering agent of the present invention, when in the presence of gastric acid, need only elevate the pH of the stomach sufficiently to achieve adequate bioavailability of the drug to effect therapeutic action.

Accordingly, examples of buffering agents include, but are not limited to, sodium bicarbonate, potassium bicarbonate, magnesium hydroxide, magnesium lactate, magnesium glucomate, aluminum hydroxide, aluminum hydroxide/sodium bicarbonate coprecipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartarate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts.

A pharmaceutically acceptable buffering agent may comprise a bicarbonate salt of Group IA metal as buffering agent, and may be prepared by mixing the bicarbonate salt of the Group IA metal, preferably sodium bicarbonate, with water. The concentration of the bicarbonate salt of the Group IA metal in the composition may be any concentration. By way of non-limiting example the bicarbonate concentration may range from approximately 5.0 percent to approximately 60.0 percent. The concentration of the bicarbonate salt of the Group IA metal may range from approximately 7.5 percent to approximately 10.0 percent. In one embodiment of the invention, sodium bicarbonate is the salt and is present in a concentration of approximately 8.4 percent.

In one embodiment, the amount of sodium bicarbonate 8.4% used in the solution of the present invention is approximately 1 mEq (or mmole) sodium bicarbonate per 2 mg lansoprazole, with a range of approximately 0.2 mEq (mmole) to 5 mEq (mmole) per 2 mg of lansoprazole.

In certain embodiments, enterically-coated lansoprazole particles may be used. Alternatively, lansoprazole powder may be used. The enterically coated lansoprazole particles may be mixed with a sodium bicarbonate (NaHCO₃) solution (8.4%), which dissolves the enteric coating and forms a lansoprazole solution. The lansoprazole solution may have pharmacokinetic advantages over standard time-released lansoprazole capsules, including: (a) more rapid drug absorbance time (about 10 to 60 minutes) following administration for the lansoprazole solution versus about 1 to 3 hours following administration for the enteric-coated pellets; (b) the NaHCO₃ solution protects the lansoprazole from acid degradation prior to absorption; (c) the NaHCO₃ acts as an antacid while the lansoprazole is being absorbed; and (d) the solution may be administered through an existing indwelling tube without clogging, for example, nasogastric or other feeding tubes Oejunal or duodenal), including small bore needle catheter feeding tubes.

Additionally, various additives may be incorporated into the inventive solution to enhance its stability, sterility and isotonicity. Further, antimicrobial preservatives, antioxidants, chelating agents, and additional buffers may be added, such as AMBICIN®. However, microbiological evidence shows that this formulation inherently possesses antimicrobial and antifungal activity. Various antibacterial and antifungal agents such as, for example, parabens, chlorobutanol, phenol, sorbic acid, and the like may enhance prevention of the action of microorganisms.

In certain embodiment, isotonic agents, for example, sugars, sodium chloride, and the like may be included in the composition. Additionally, thickening agents such as methylcellulose may be desirable to use in order to reduce the settling of the lansoprazole or other PPI or derivatives thereof from the suspension.

A solution may further comprise flavoring agents (e.g., chocolate, root beer or watermelon) or other flavorings stable at pH 7 to 9, and anti-foaming agents (e.g., simethicone 80 mg, Mylicon™).

Embodiments of the invention further includes a pharmaceutical composition including lansoprazole or other proton pump inhibitor and derivatives thereof and one or more buffering agents in a form convenient for storage, whereby when the composition may be placed into an aqueous solution, the composition dissolves yielding a suspension suitable for enteral administration to a subject. The pharmaceutical composition may be in a solid form prior to dissolution or suspension in an aqueous solution. The lansoprazole or other PPIs and buffering agent may be formed into a tablet, capsule, pellets or granules, by methods well known to those skilled in the art.

The resultant lansoprazole solution may be stable at room temperature for several weeks and inhibit the growth of bacteria or fungi. The solution may maintain greater than 90% of its potency for 12 months. By providing a pharmaceutical composition including lansoprazole or other PPI with buffer in a solid form, which may be later dissolved or suspended in a prescribed amount of aqueous solution to yield the desired concentration of lansoprazole and buffer, the cost of production, shipping, and storage may be greatly reduced as no liquids are shipped (reducing weight and cost), and there is no need to refrigerate the solid form of the composition or the solution. Once mixed, the resultant solution may then be used to provide dosages for a single patient over a course of time, or for several patients.

As mentioned above, the formulations of the present invention may also be manufactured in concentrated forms, such as tablets, suspension tablets and effervescent tablets or powders, such that upon reaction with water or other diluent, an aqueous form of the present invention is produced for oral, enteral, or parenteral administration.

In addition to the suspension tablet, the solid formulation of the present disclosure may be in the form of a powder, a tablet, a capsule, or other suitable solid dosage form (e.g., a pelleted form or an effervescing tablet, troche or powder), which creates a solution according to the invention in the presence of diluent or upon ingestion. For example, the water in the stomach secretions or water which is used to swallow the solid dosage form may serve as an aqueous diluent.

The pharmaceutical compositions of the present disclosure include a PPI, for example lansoprazole, as the active ingredient, or a pharmaceutically acceptable salt thereof, and may also include a pharmaceutically acceptable carrier, and optionally, other therapeutic ingredients.

According to an embodiment of the invention, a sodium salt, a lithium salt, a potassium salt, a magnesium salt, a calcium salt, or a barium salt of lansoprazole may be used. The salt may be a crystal. For example, the salt of the present invention may be a sodium salt (in particular a crystal) of (R)-2-[[[3-methyl-4-(2,2,2-trifluoroethoxy)-2-pyridinyl]methyl]sulfinyl]-1H-benzimidazole, a potassium salt (in particular a crystal) of (R)-2-[[[3-methyl-4-(2,2,2-trifluoroethoxy)-2-pyridinyl]methyl]sulfinyl]-1H-benzimidazole, and the like. Further, the salt may be solvated.

In one embodiment of the invention, the PPI may be administered in combination with PEG 300. The active ingredient, for example lansoprazole, may be mixed with PEG 300 and administered to a subject.

In one embodiment of the invention, the PPI, e.g., lansoprazole, may be administered to a subject together with another chemotherapeutic agent. Other contemplated chemotherapeutic agents include, but are not limited to, alkylating agents, such as carboplatin and cisplatin; nitrogen mustard alkylating agents; nitrosourea alkylating agents, such as carmustine (BCNU); antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as aldesleukin, interleukin-2, docetaxel, etoposide (VP-16), interferon, paclitaxel, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine. Further, the following additional drugs may also be used in combination with the PPI and/or another chemotherapeutic agent, even if not considered antineoplastic agents themselves: dactinomycin; daunorubicin HCl; docetaxel; doxorubicin HCl; epoetin alfa; etoposide (VP-16); ganciclovir sodium; gentamicin sulfate; interferon leuprolide acetate; meperidine HCl; methadone HCl; ranitidine HCl; vinblastin sulfate; and zidovudine (AZT).

The phrase “combination therapy,” as used herein, refers to co-administering a PPI inhibitor, for example, lansoprazole, and another chemotherapeutic agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

Combination therapy may also embrace the administration of the therapeutic agents as described above in further combination with other biologically active ingredients and non-drug therapies. Where the combination therapy further includes a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination may be administered to a patient simultaneously or sequentially. It will be appreciated that the components may be present in the same pharmaceutically acceptable carrier and, therefore, are administered simultaneously. Alternatively, the active ingredients may be present in separate pharmaceutical carriers, such as, conventional oral dosage forms, that can be administered either simultaneously or sequentially.

According to one embodiment of the invention, a PPI may be used in the manufacture of a medicament for the treatment of a tumor. For example, use of lansoprazole in the manufacture of a medicament for the treatment of a tumor represents one embodiment of the invention.

The present invention is further described in the following examples, which are offered by way of illustration and are not intended to limit the invention in any manner.

### EXAMPLE I

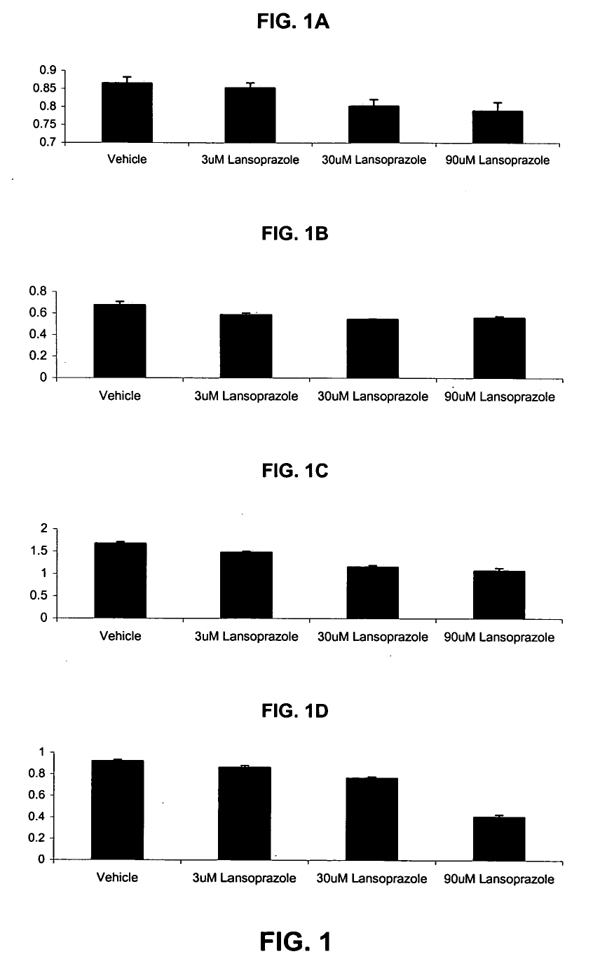

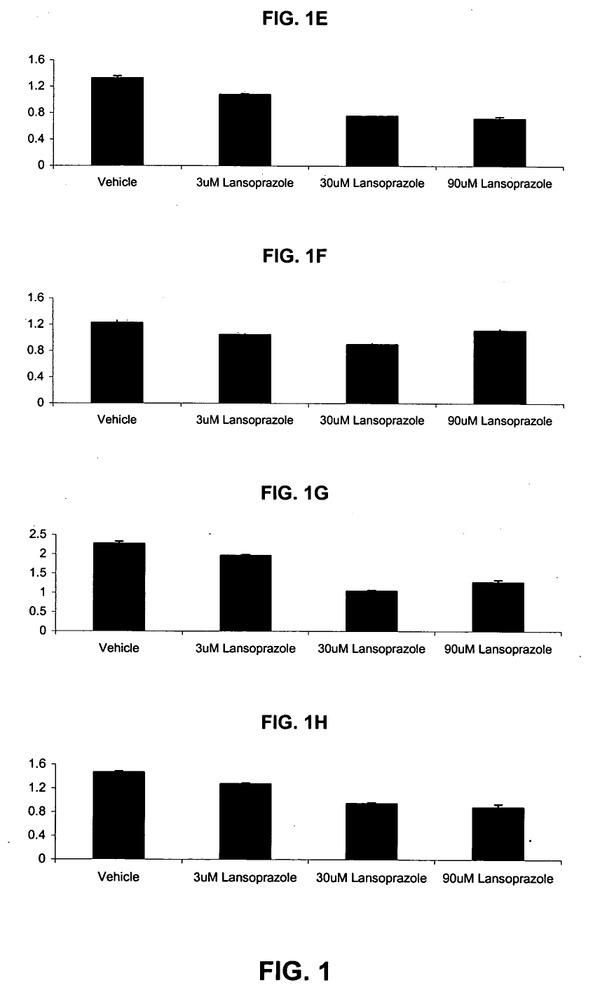

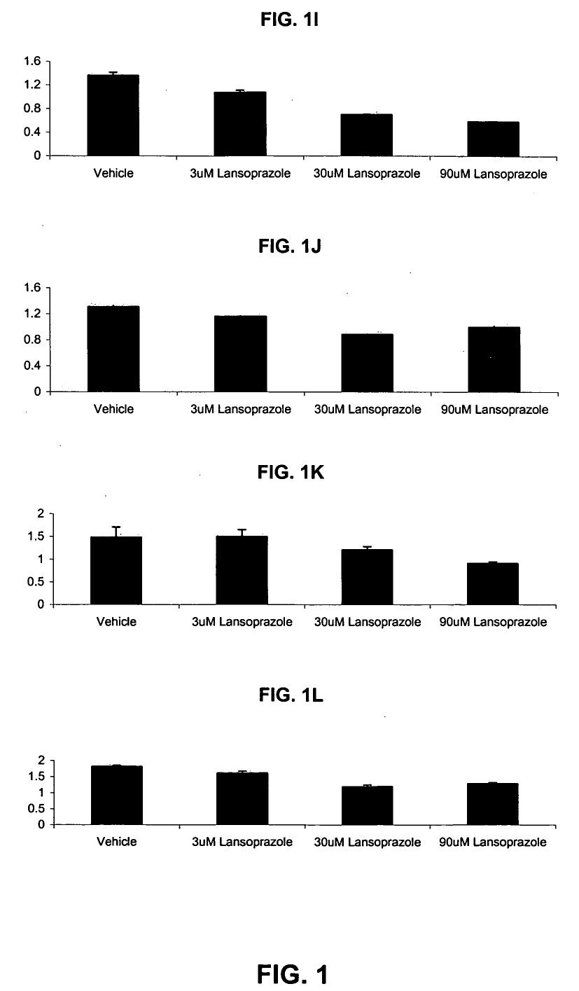

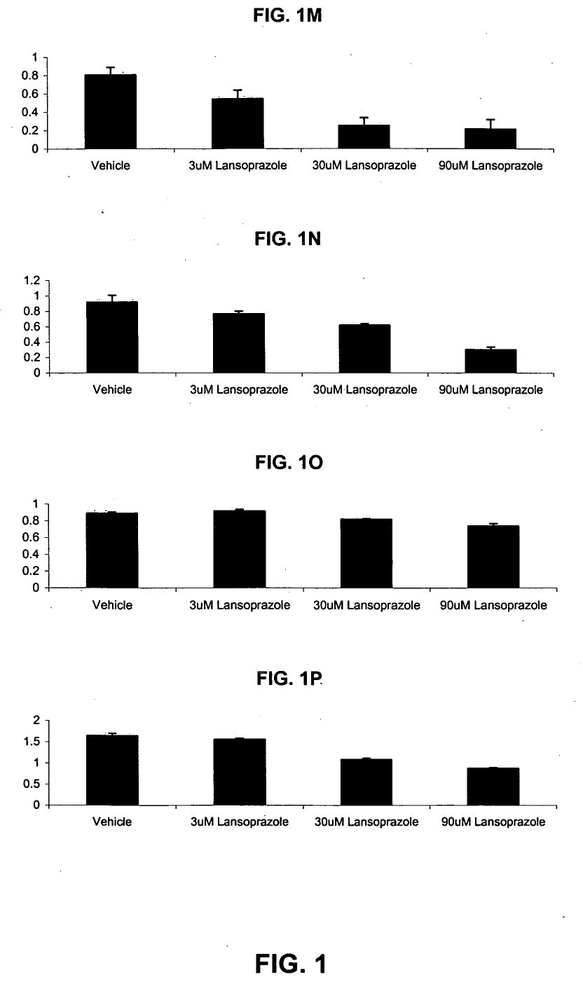

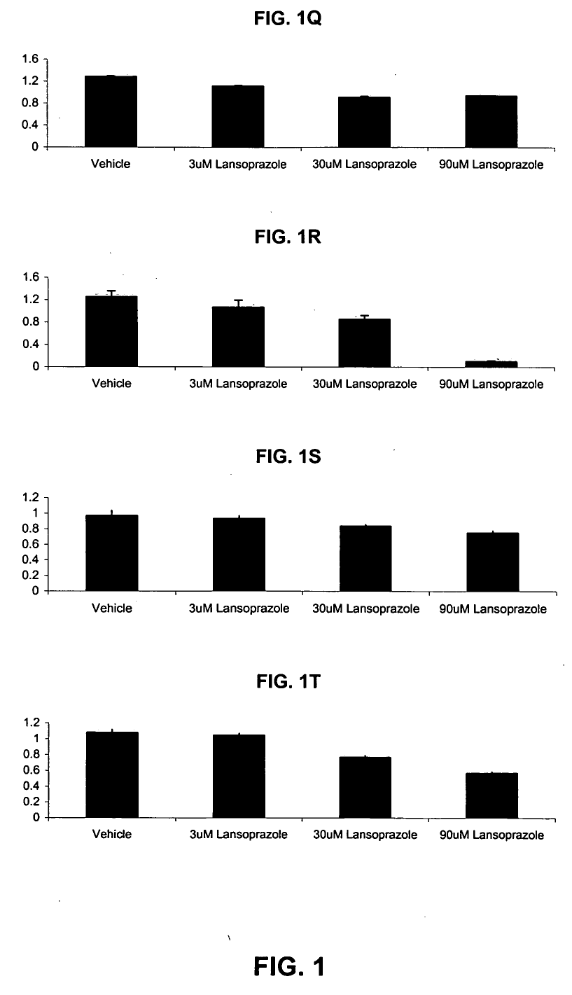

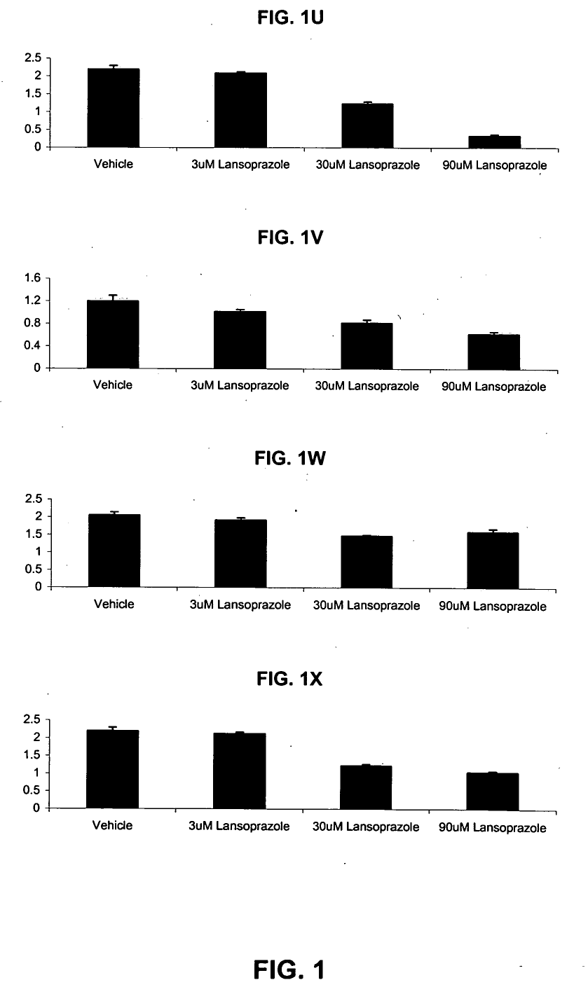

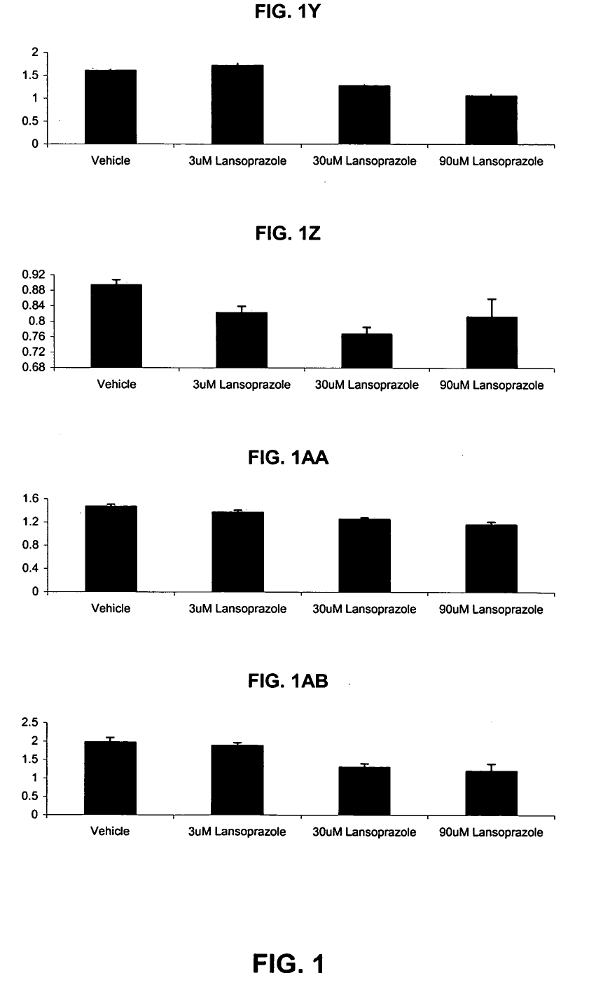

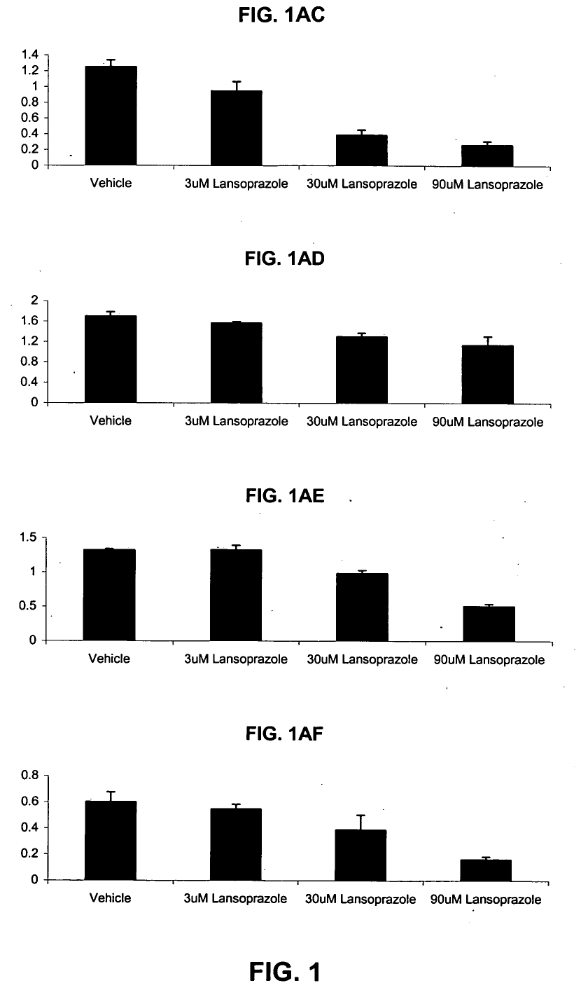

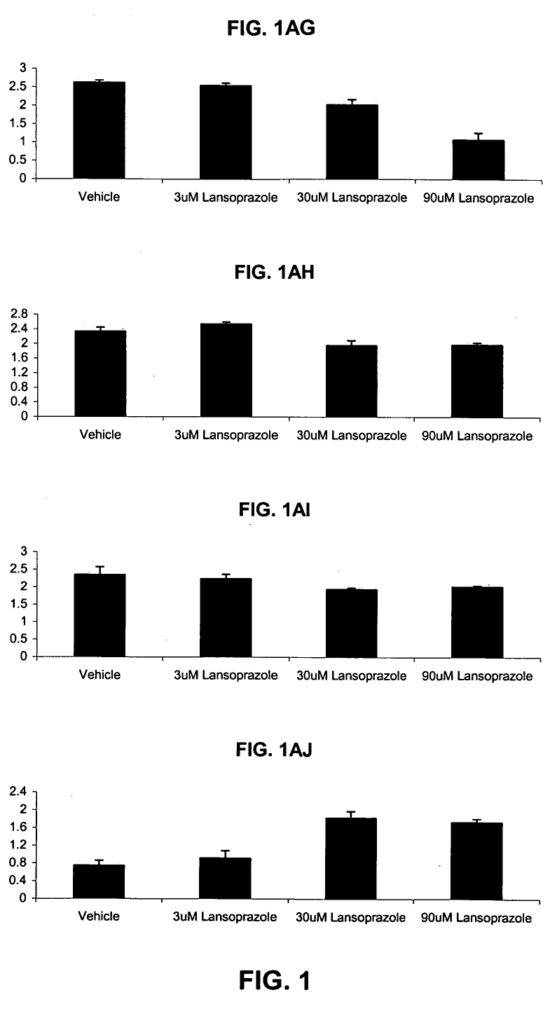

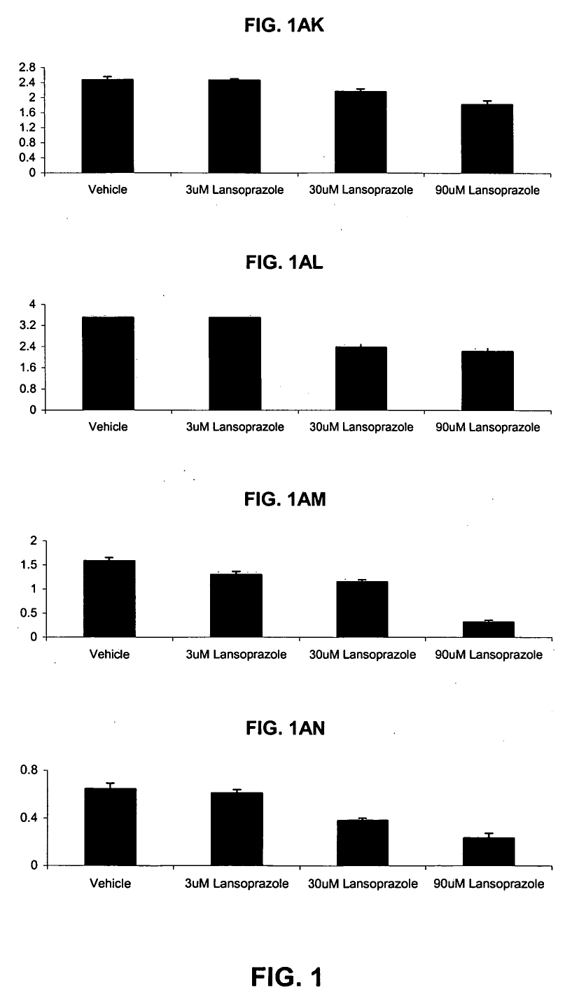

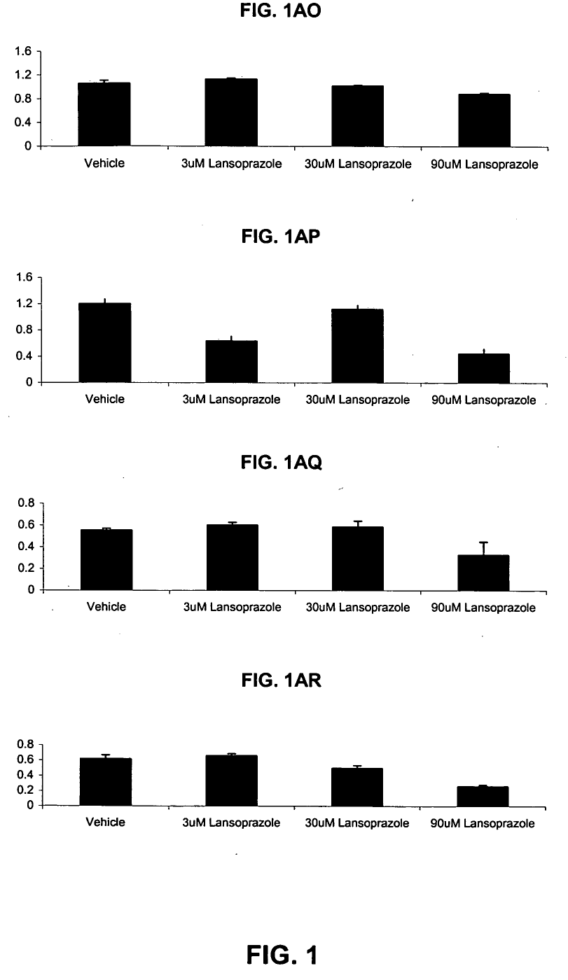

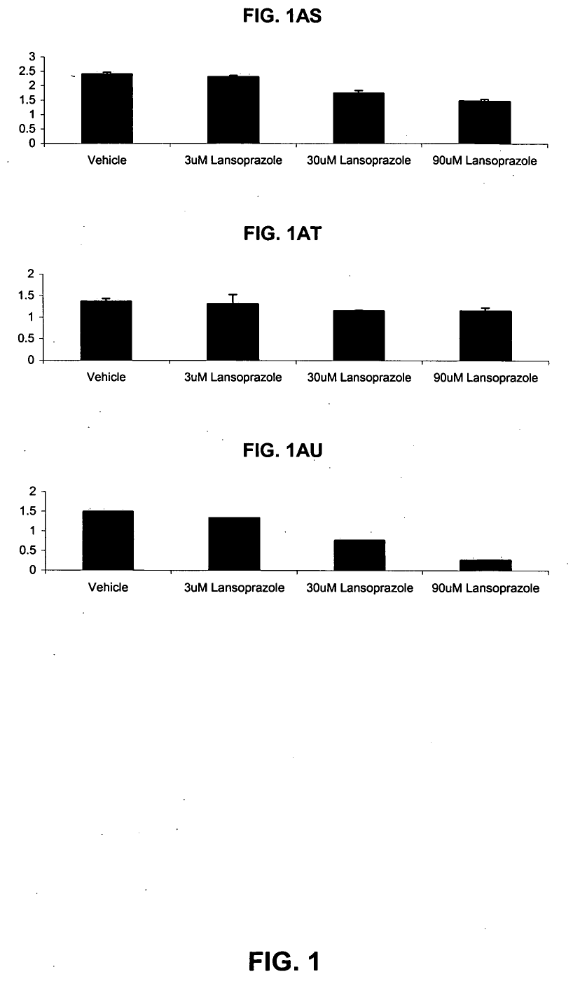

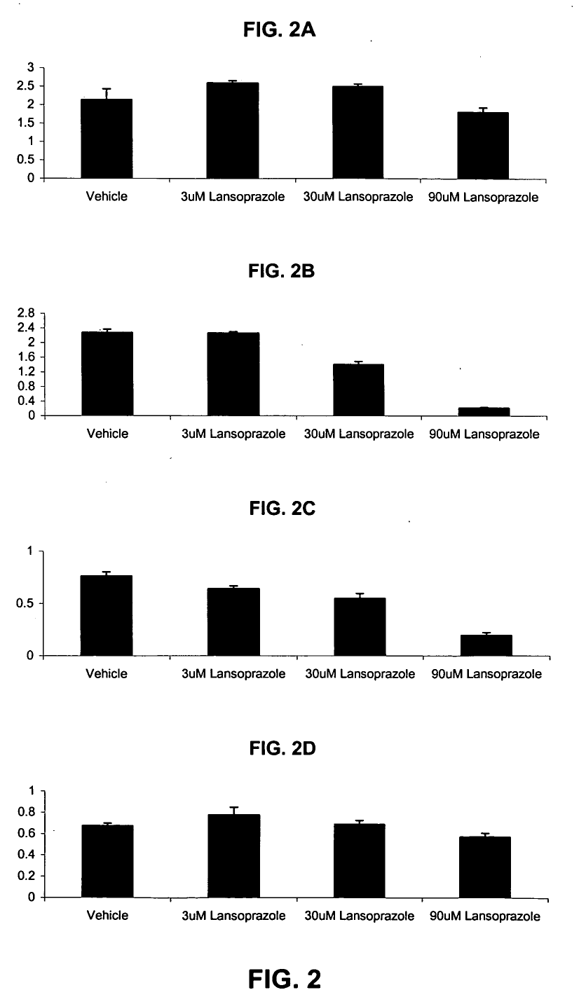

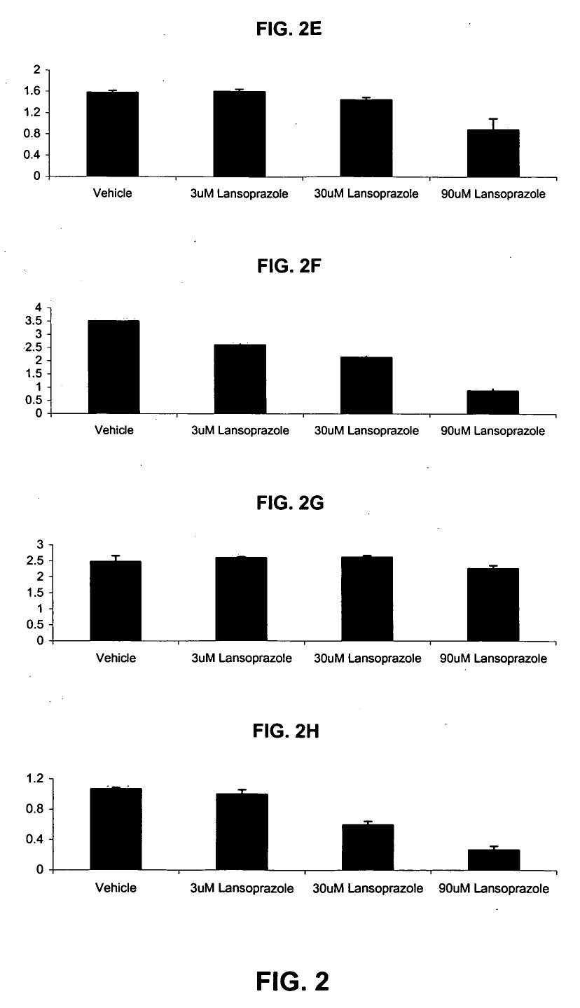

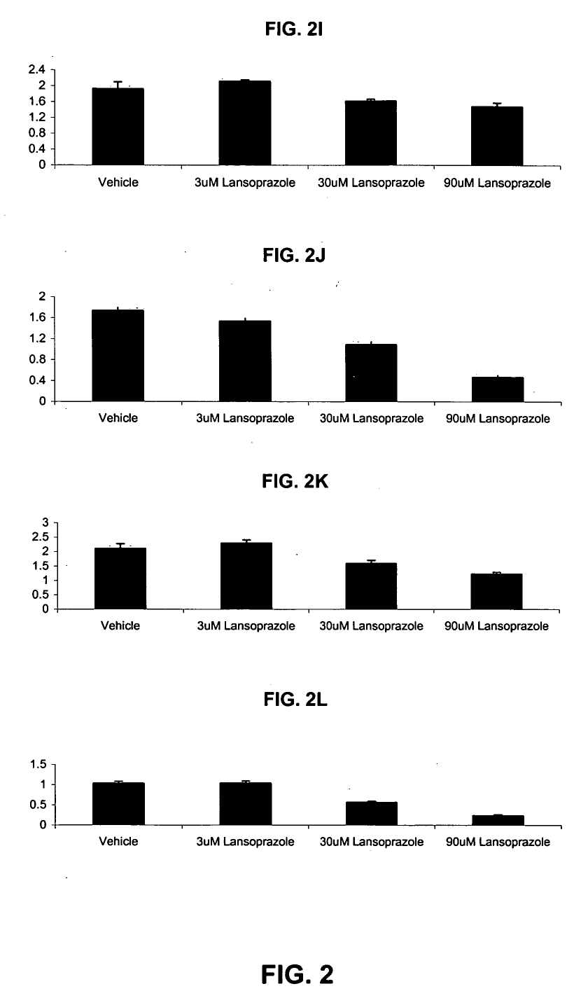

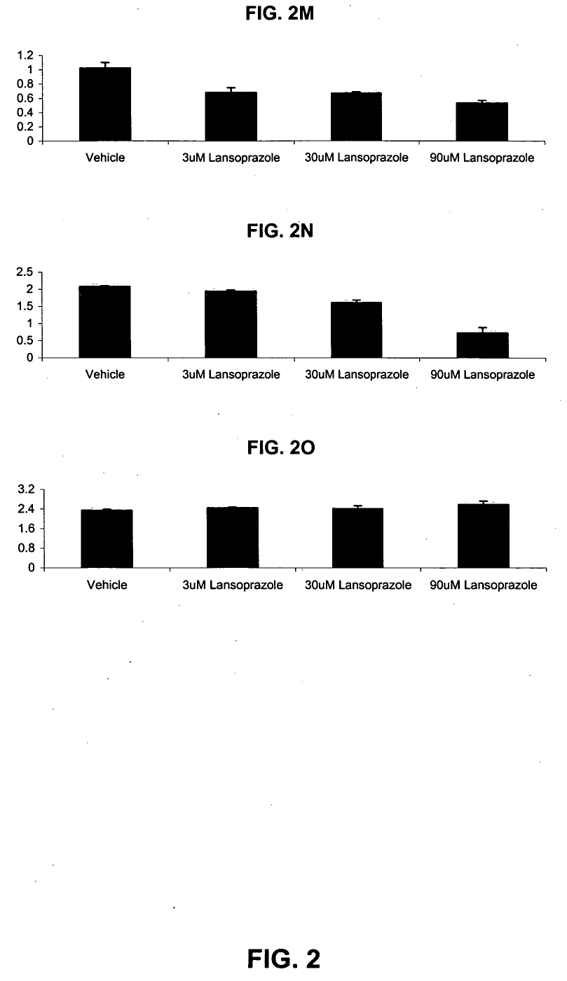

In one study, adherent cells were plated approximately 16-24 hours before the day of the experiment in 180 μl growth media. On the day of the experiment, the plated adherent cells were analyzed and counted and suspension cells were plated in 180 μl growth media. 10× lansoprazole compounds and vehicle was prepared. The 10× lansoprazole compounds were prepared by diluting lansoprazole to final concentrations of 100 μM, 30 μM, 10 μM, 3 μM, and 1 μM. A vehicle was prepared by preparing a solution of PBS, equivalent to the volume used for 10 μM lansoprazole wherein the 1× solution constitutes ˜0.06% PBS. 10 μl of the various 10× lansoprazole solutions and the vehicle were added to the cells. The cells were incubated at 37° C., 5% CO₂ for 48 hours. The media was then aspirated. The cell suspension was then spun at 1500 RPM for 10 minutes. The media was slowly removed. 200 μl of MTT solution was added to each well with a concentration of 0.863 mg/ml MTT in the growth media. The cells were incubated at 37° C., 5% CO₂ for 4 hours and the medium is then aspirated. The plate containing the cell suspension was then spun at 1500 RPM for 10 minutes. The media was slowly removed using a multichannel pipettor. 100 μl of DMSO was then added to each well. The cells were incubated at 37° C., 5% CO₂ for 5 minutes and the absorbance was obtained at 560 nm using a Dynex Opsys MR plate reader. The cells used for this procedure were the following cell lines: IGROV1 (ovarian cancer); OVCAR 8 (ovarian cancer); ES-2 (ovarian cancer); CAOV3 (ovarian cancer); OVCAR5 (ovarian cancer); SUDHM (lymphoma); RPM18226 (myeloma); RPMI6666 (Hodgkin's lymphoma); NC37 (lymphoma); GDM-1 (leukemia); MC/CAR (prostate cancer); DU-145 (prostate cancer), J45-01 (leukemia), MOLT3 (leukemia), HUT78, J-gamma-1 (leukemia), MCF7 (leukemia), COLO205 (colon cancer), HL60 CLONE 15 (leukemia), T47D (breast cancer), P116 (intestinal cancer), KU812 (leukemia), SW620 (colon cancer), MK92-M1 (lymphoma), MV-4-11 (leukemia), HS578T (breast cancer), HT29 (colon cancer), HCT 116 (colon cancer), CAKI-1 (kidney cancer), A549 (lung cancer), H460 (lung cancer), A3 (colon cancer), MDA-MB231 (melanoma), MALME 3M (melanoma), RXF393 (kidney cancer), RS4.11 (leukemia), PC3 (prostate cancer), OVCAR3 (ovarian cancer), MDA-MB435 (melanoma), HUT102 (lymphoma), HL60 (leukemia), CCRF-CEM (leukemia), HUT78 (lymphoma), 786-O (kidney cancer), ACHN (kidney cancer), A498 (kidney cancer), H226 (lung cancer), H522 (lung cancer), HOP92 (lung cancer), SNB 19 (brain cancer), OVCAR4 (ovarian cancer), H9 (lymphoma), UO-31 (kidney cancer), HH (lymphoma), DAUDI (leukemia), LOXIMVI (melanoma), NAMALWA (lymphoma), EKVX (lung cancer), DOHH2 (lymphoma), SNB75 (brain cancer), SKMEL28 (melanoma), SKMEL5 (melanoma), SKMEL2 (melanoma), M14 (melanoma), UACC 257 (melanoma), H332M (lung cancer), KM12 (colon cancer), HCC2998 (colon cancer), G401 (kidney cancer), RS 1184 (lymphoma), MC116 (leukemia), MOLT4 (leukemia), JM1 (liver cancer), HOP-62 (lung cancer), HCT-15 (colon cancer), SF-539 (brain cancer), SF295 (brain cancer), ST486 (lymphoma), U251 (brain cancer), and UACC-62 (melanoma). As shown in FIGS. 1A-AU, 2A-2O, 6A-6M the following cell lines exhibited significant tumor cell growth reduction: ES-2, IGROV 1, OVCAR5, OVCAR8, J-gamma-1, KU812, NK92MI, 786-O, A498, H522, SNB19, OVCAR4, H9, HH, EKVX, OVCAR5, UACC257, H226, UO-31, NAMALWA, SKMEL28, SKMEL2, M14, H322M, HCC2998, HL60, HT29, A549, RXF393, PC3, H460, RPM18226, NC37, MC/CAR, SUDHLA, RPMI6666, GDM-1, MOLT3, J45-01, MCF7, HL60 clone 15, P116, SW620, MV-4-11, SKMEL5 (melanoma), DAUDI, DOHH2, HUT102, CCRF-CEM, HUT78, A3, MDA-MB-435, MDA-MB-231, RS4.11, MC116, MOLT4, JMI, HOP-62, HCT-15, SF-539, SF295, ST486, U251, and UACC-62.

### EXAMPLE II

Adherent cells are plated approximately 16-24 hours before the day of the experiment in 180 μl growth media. On the day of the experiment, the plated adherent cells are analyzed and counted and suspension cells are plated in 180 μl growth media. The 10× lansoprazole compound, vehicle and chemotherapeutic cocktail are prepared. 10× lansoprazole compounds are prepared by diluting lansoprazole to final concentrations of 100 μM, 30 μM, 10 μM, 3 μM, and 1 μM. A vehicle is prepared by preparing a solution of PBS, equivalent to the volume used for 10 μM lansoprazole wherein the 1× solution constitutes ˜0.06% PBS. The chemotherapeutic cocktail is prepared by obtaining a 10× solution by diluting a Velcade solution to 10 μM, 100 μM, a Etoposide solution to 1 mM and 20 μM, and a Taxol solution to 200 μM. 10 μl of the various 10× lansoprazole solutions, the chemotherapeutic cocktail and the vehicle are added to the cells. The cells are incubated at 37° C., 5% CO₂ for 48 hours. The media is then aspirated. The plate containing the cell suspension is then spun at 1500 RPM for 10 minutes. The media is slowly removed using a multichannel pipettor. 200 μl of MTT is added to each well to a concentration of 0.863 mg/ml MTT in the growth media. The cells are incubated at 37° C., 5% CO₂ for 4 hours and then aspirated. The plate containing the cell suspension is then spun at 1500 RPM for 10 minutes. The media is slowly removed using a multichannel pipettor. 100 μl of DMSO is added to each well. The cells are incubated at 37° C., 5% CO₂ for 5 minutes and the absorbance is obtained at 560 nm using a Dynex Opsys MR plate reader. Cells used for this procedure may include the following cell lines: IGROV1, OVCAR 8, ES-2, CAOV3, OVCAR5 SUDHL4, RPM18226, RPMI6666, NC37, GDM-1, MC/CAR, DU-145, J45-01, MOLT3, HUT78, J-GAMMA1, MCF7, COLO205, HL60 CLONE 15, T47D, P116, KU812, SW620, MK92-M1, MV-4-11, HS578T, HT29, HCT 116, CAKI-1, A549, H460, A3, MDA-MB231,MALME 3M, RXF393, RS4.11, PC3, OVCAR3, MDA-MB435, HUT102, HL60, CCRF-CEM, HUT78, 786-0, ACHN, A498, H226, H522, HO P92, SNB 19, OVCAR4, H9, UO-31, HH, DAUDI, LOXIMVI, NAMALWA, EKVX, DOHH2, SNB75, SKMEL28, SKMEL5, SKMEL2, M14, UACC 257, H332M, KM12, HCC2998, G401, and RS1184.

### EXAMPLE III

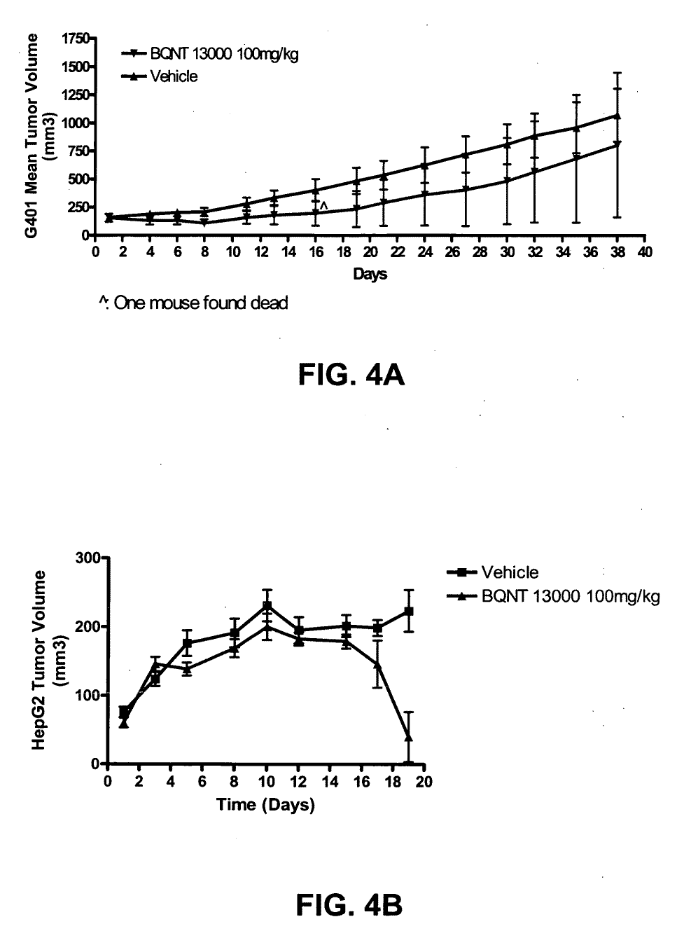

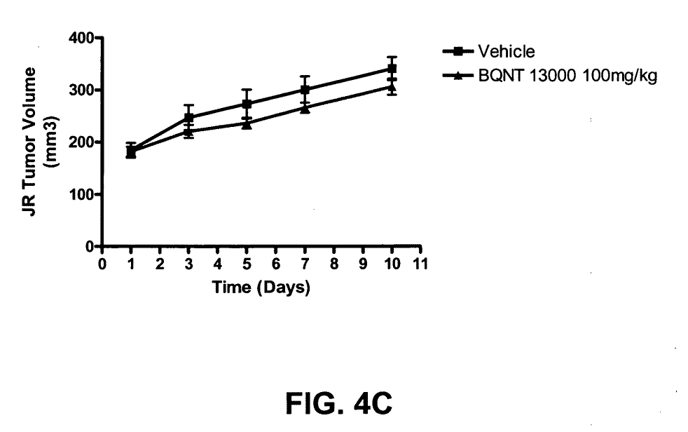

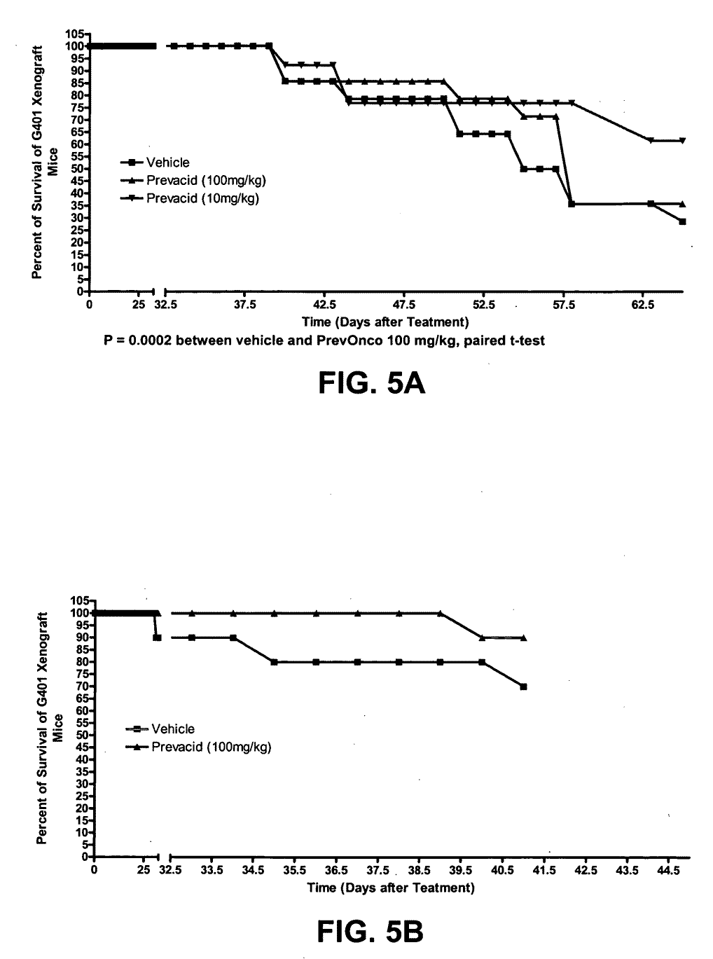

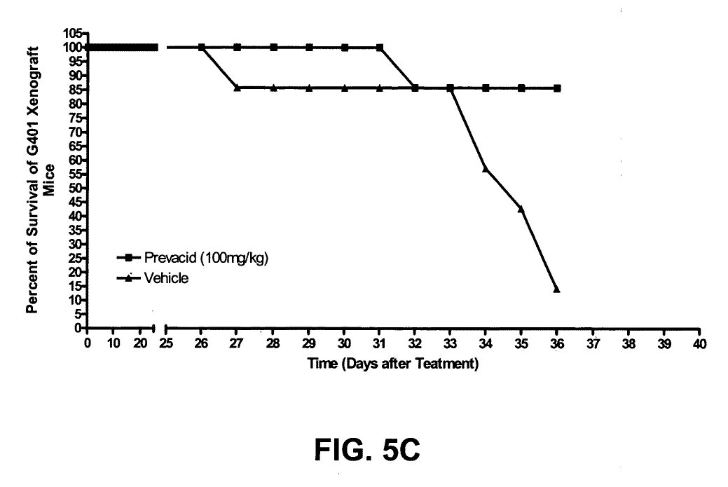

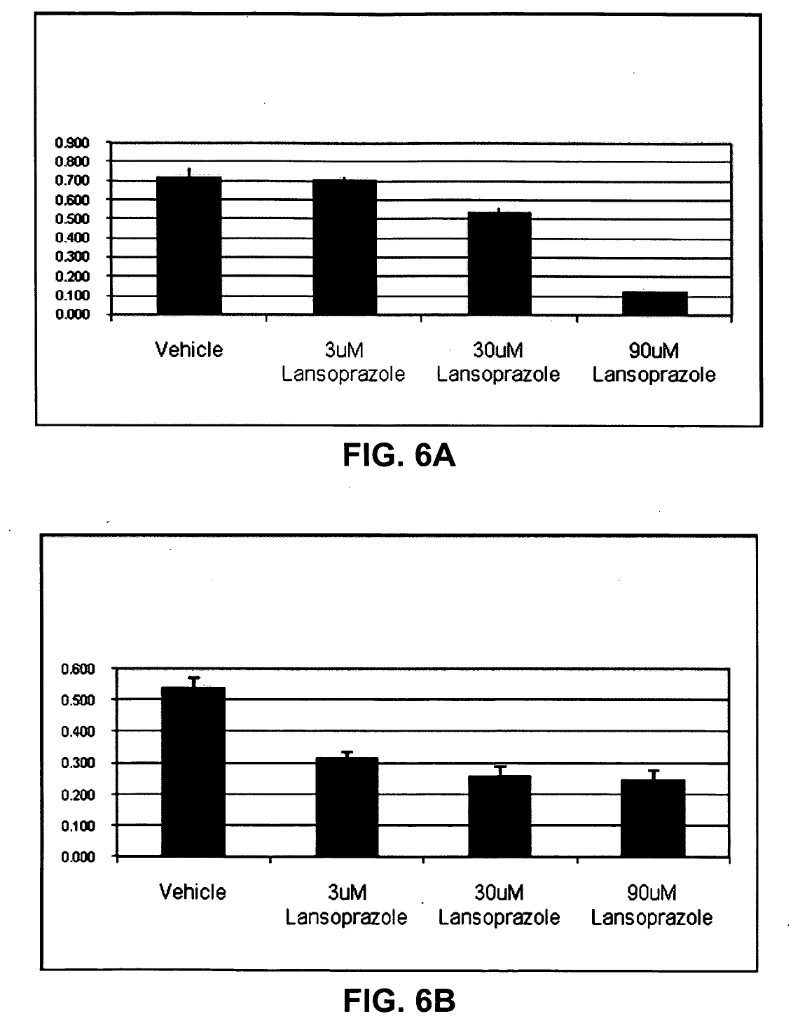

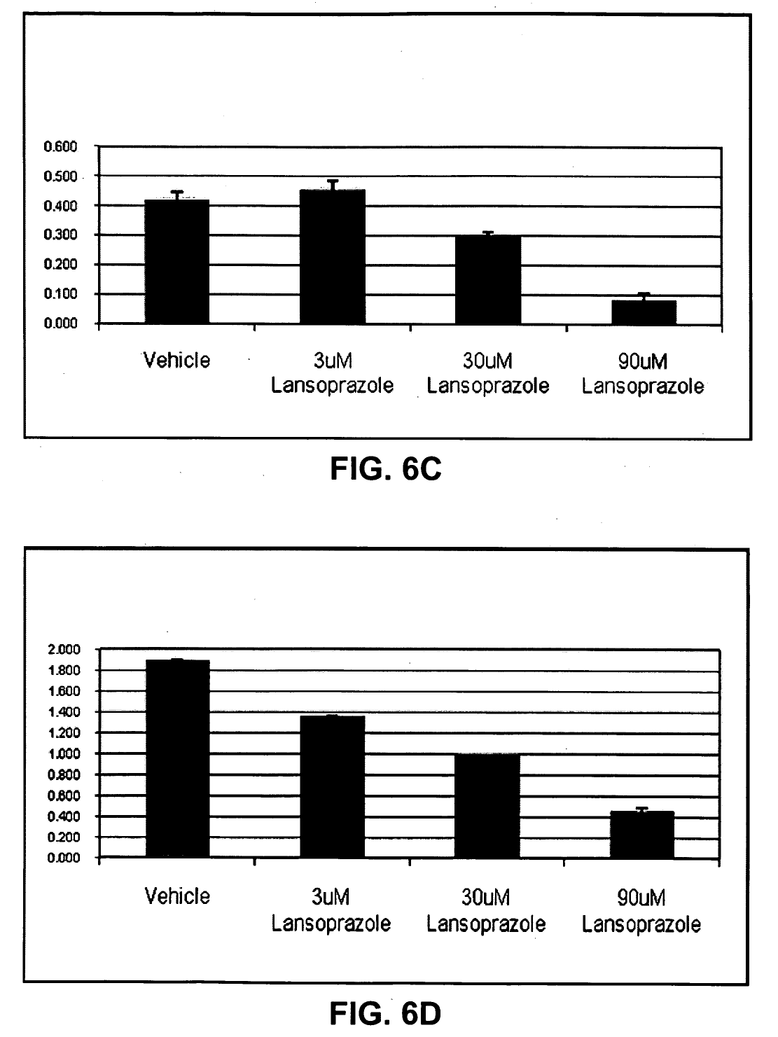

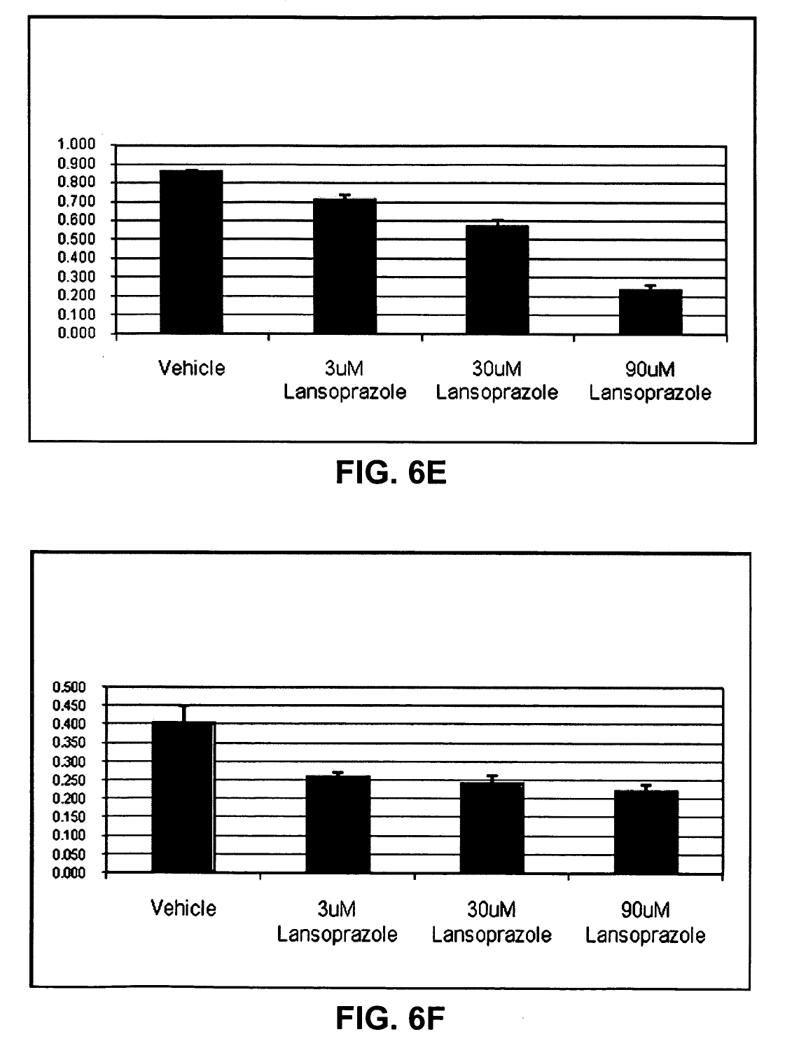

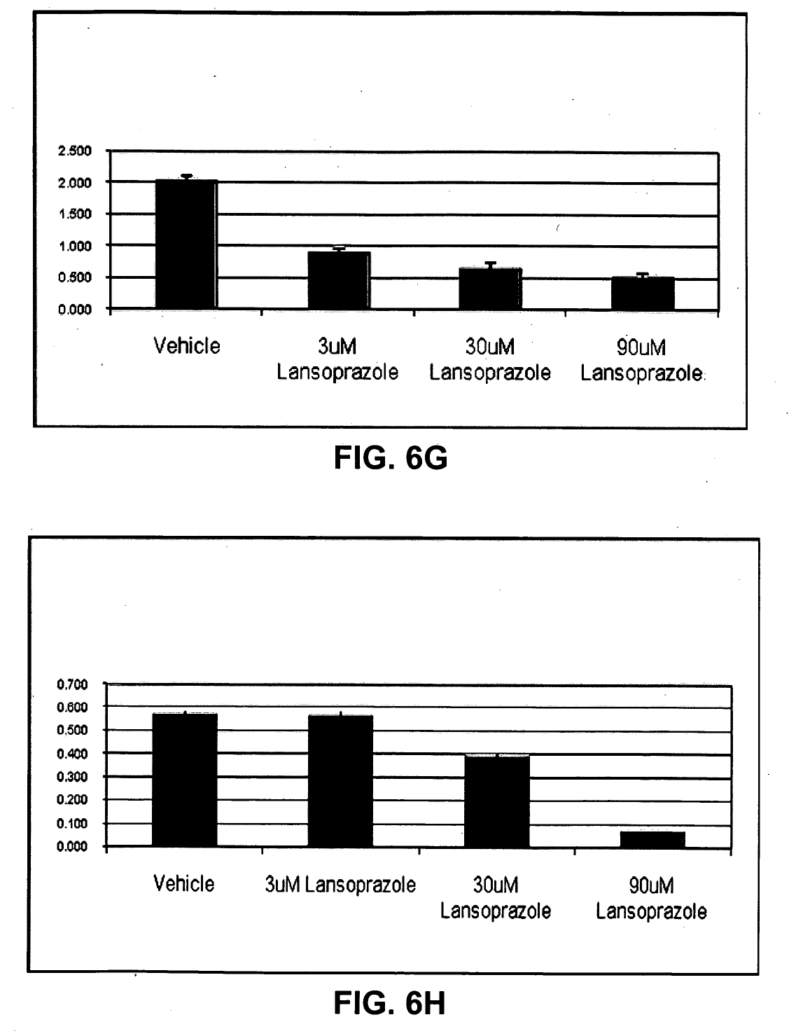

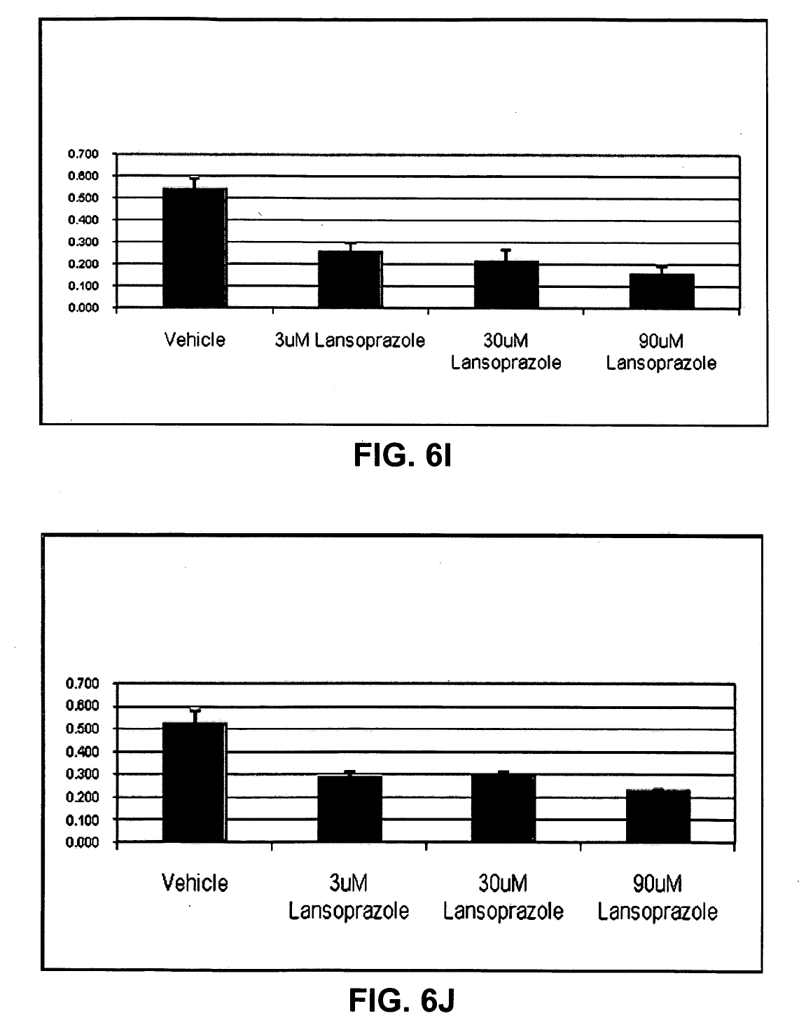

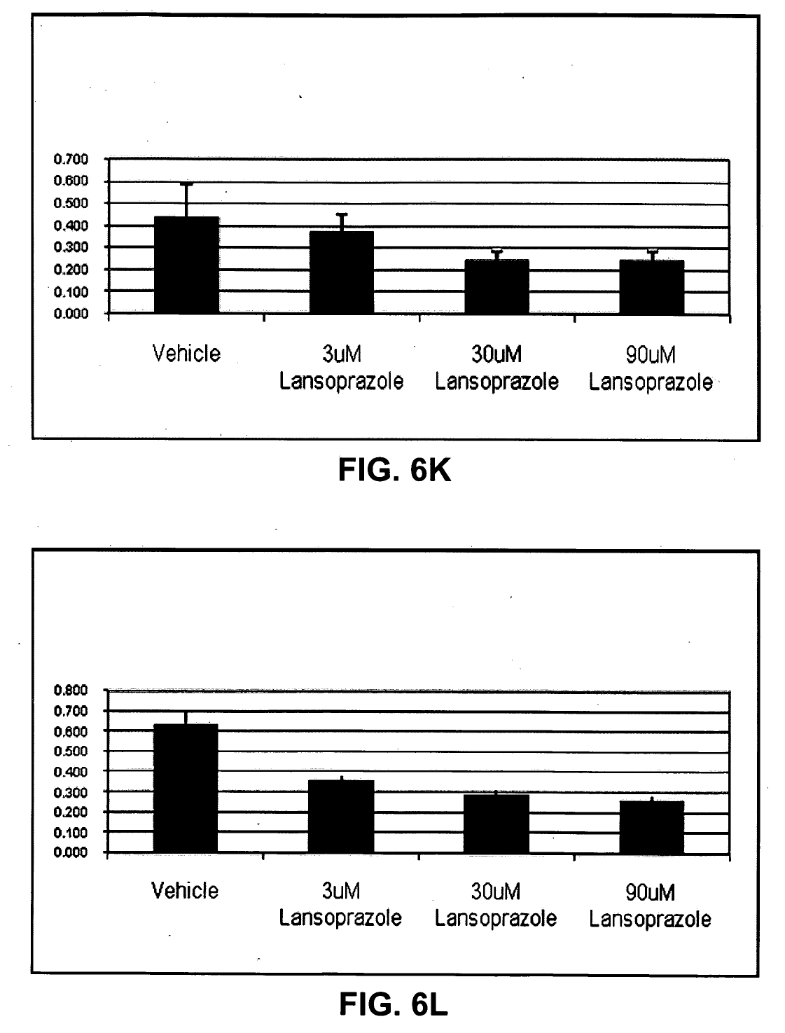

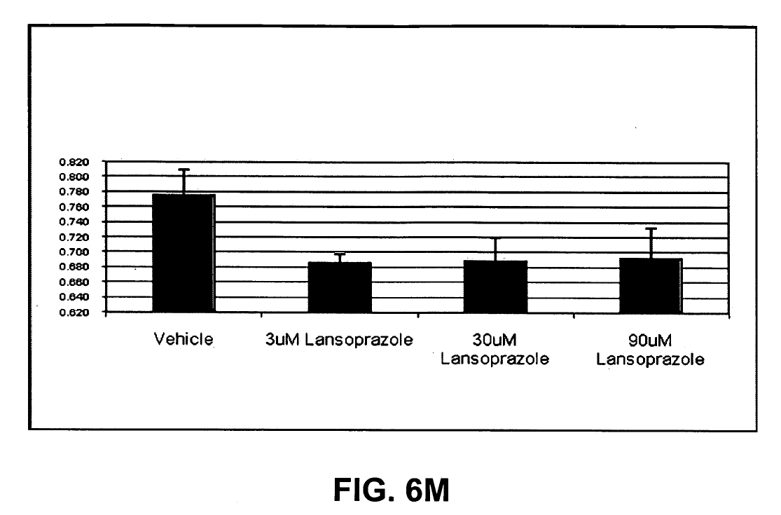

JR (rhabdomyosarcoma), HepG2 (liver carcinoma), SR Liquid Leukemia (leukemia), RS1184B Lymphoma (lymphoma), and G401 rhabdoid (kidney) cancer cells for use in a xenograft mouse model were collected from JR, HepG2, SR Liquid Leukemia, RS1184 B Lymphoma, and G401 rhabdoid tumor cell lines and injected at 5×10⁶ cells per nude mouse in a volume of 100 μl subcutaneously. The animals were examined three times weekly to determine the progression of the tumors and determine body weight. Dosing started when tumors reached the ˜75-150 mm³ size. Animals were randomized and distributed in groups in such a way that mean tumor weights in all groups were within 15% of the mean tumor weight in Group 1 (Control-vehicle group). Upon reaching the ˜75-150 mm³ target size an effective amount of lansoprazole was administered to the mice in a suspension of PEG300 at a concentration of 100 mg/kg. As indicated in FIGS. 4A-4C and 5A-5C, lansoprazole effectively reduced tumor growth in mice and extended life span of the population. Lansoprazole was especially effective in treating tumor cells derived from the HepG2, G401, and SR Liquid Leukemia tumor cell lines.

### EXAMPLE IV

Experiments, as described in Example II and Example III, are conducted with ATPase inhibitors such as omeprazole, lansoprazole, pantoprazole, rabeprazole, dontoprazole, perprazole (s-omeprazole magnesium), habeprazole, ransoprazole, pariprazole, and leminoprazole.

### EXAMPLE V

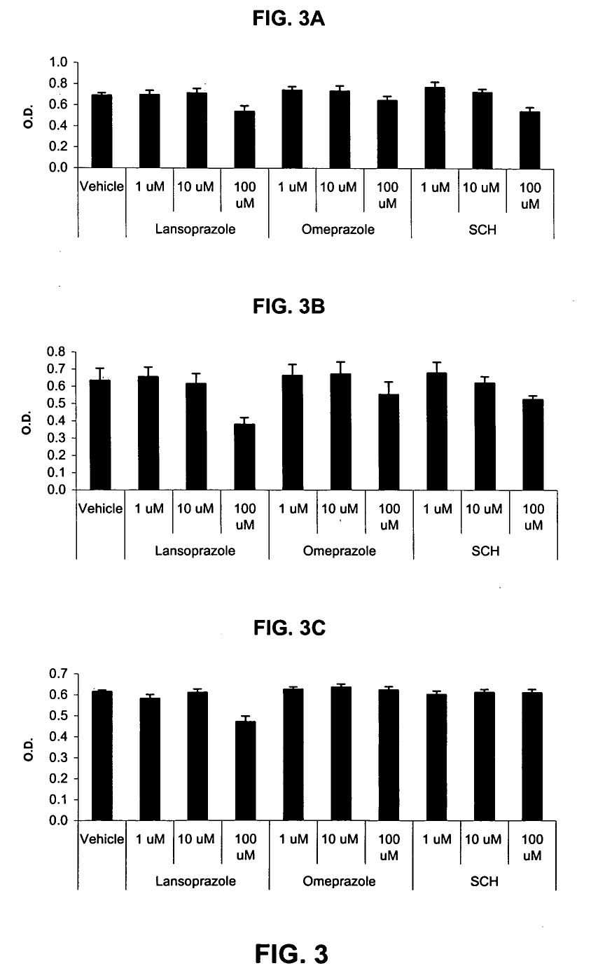

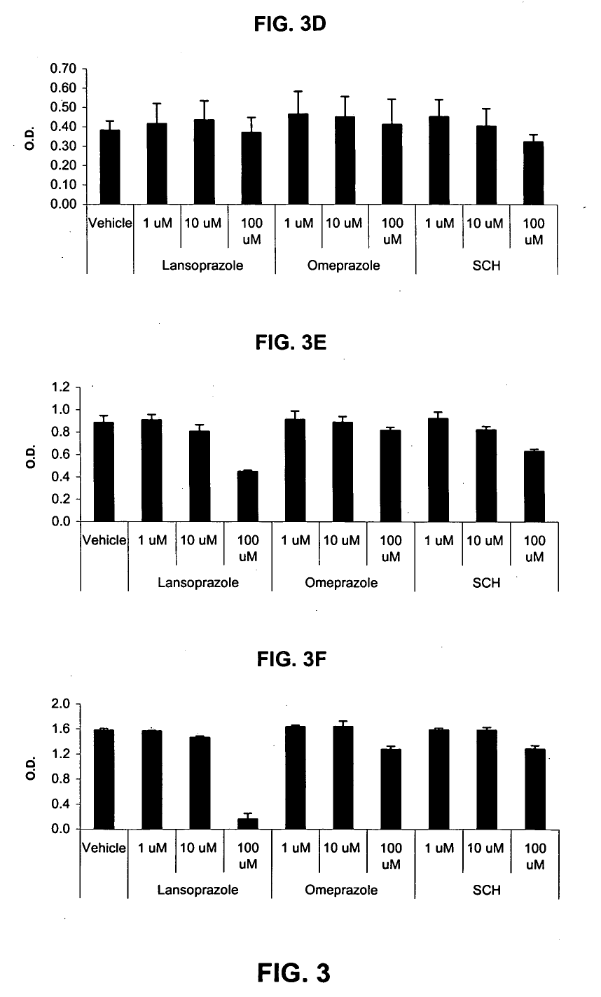

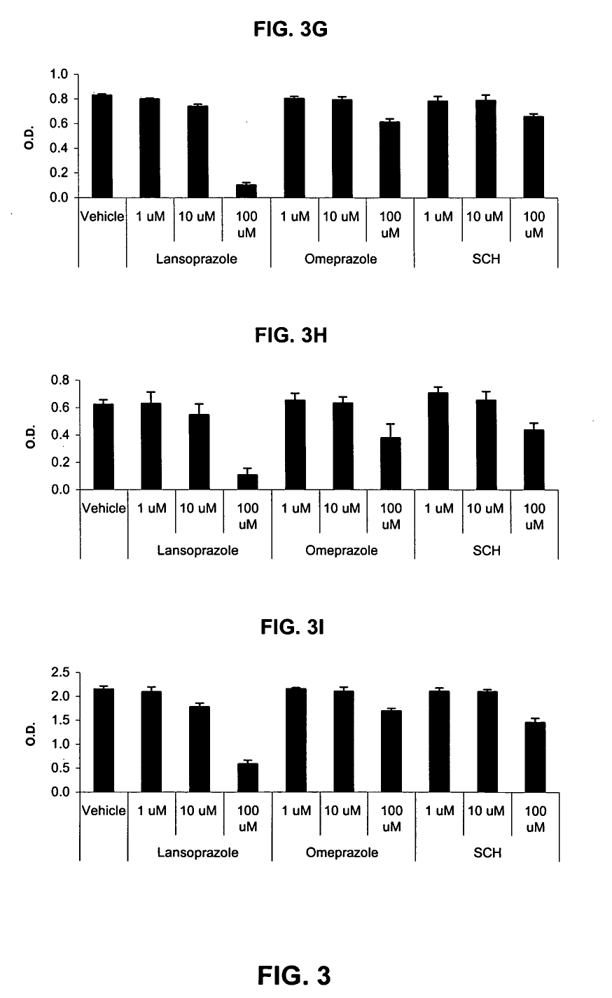

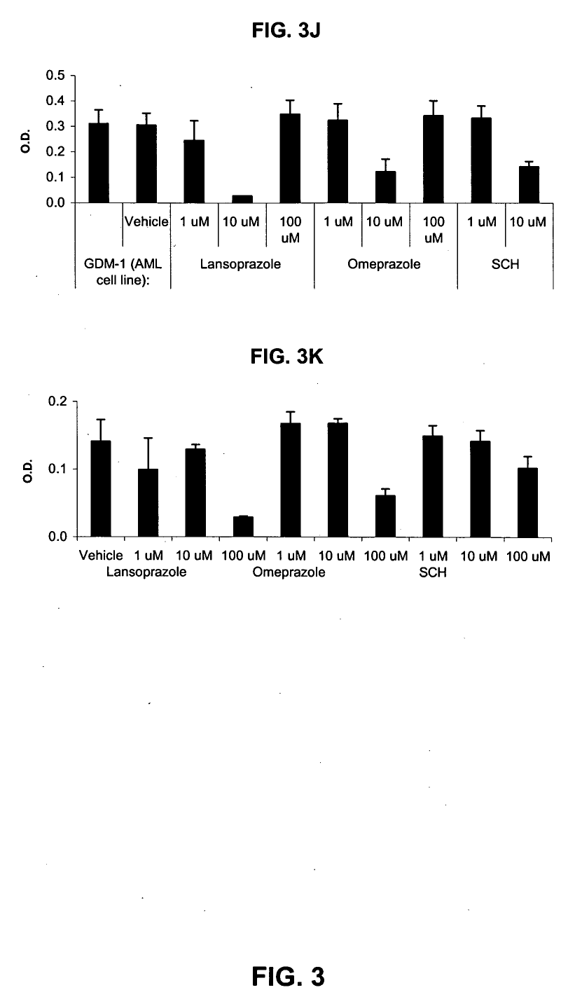

Experiments, as described in Example 1 were conducted with lansoprazole, omeprazole and SCH 28080 at concentrations of 1 μM, 10 μM, and 100 μM. FIGS. 3A-3K show the experimental results when tumor cells from the following cell lines were treated with lansoprazole, omeprazole, and SCH 28080: IGROV1 (FIG. 3A), OVCAR8 (FIG. 3B), ES-2 (FIG. 3C), CAOV3 (FIG. 3D), OVCAR5 (FIG. 3E), SUDHL4 (FIG. 3F), RPMI8226 (FIG. 3G), RPMI6666 (FIG. 3H), NC37 (FIG. 31), GDM-1 (FIG. 3J), and MC/CAR (FIG. 3K). As can be seen in FIGS. 3E-I and 3K 100 μM of lansoprazole was effective in reducing tumor cell growth (Omeprazole was effective for reducing tumor cell growth for the MC/CAR cell line as depicted in FIG. 3K). Additionally, a 10 μM concentration of lansoprazole, omeprazole, and SCH28080 was effective in reducing tumor cell growth of the GDM-1 cell line as depicted in FIG. 3J.

### EXAMPLE VI

In order to reduce the size of a tumor in a subject, the subject is first examined and diagnosed with a tumor. Upon diagnosis of the tumor, a pharmaceutically acceptable composition is administered to the subject. The pharmaceutically acceptable composition contains lansoprazole, an excipient, and instructions for administering the pharmaceutically acceptable composition to the subject suffering from the tumor. It is determined that the subject is not suffering from elevated gastric acid production. The pharmaceutically acceptable composition is administered according to the instructions provided. The instructions indicate how much of the pharmaceutically acceptable composition is administered to the subject in order to reduce the size of the diagnosed tumor. Upon appropriate administration the volume of the diagnosed tumor is reduced, further growth of the tumor is inhibited via cellstatic activity, and tumor cells are killed by the pharmaceutically acceptable composition via cellcidal activity.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### EQUIVALENTS

It is understood that the disclosed invention is not limited to the particular methodology, protocols, and dosages described as these may vary. It is also to be understood that the terminology used herein is for the purpose of describing particular embodiments only, and is not intended to limit the scope of the present invention which will be limited only by the appended claims.

### INCORPORATION BY REFERENCE

The entire disclosure of each of the patent documents and scientific articles referred to herein is incorporated by reference for all purposes

1. A method of killing tumor cells, the method comprising:
administering a pharmaceutically acceptable composition comprising an effective amount of a proton pump inhibitor or a pharmaceutically acceptable salt thereof to the tumor cells so as to decrease tumor volume.

2. A method of reducing the size of a tumor in a subject, the method comprising:
diagnosing the tumor in the subject; and
administering to the subject a pharmaceutically acceptable composition comprising a proton pump inhibitor in an amount sufficient to reduce the size of the tumor.

3. The method of claim 1, wherein upon administration the pharmaceutically acceptable composition interacts with tumor cells outside of the gastric lumen.

4. The method of claim 1, further comprising selecting the proton pump inhibitor from the group consisting of lansoprazole, omeprazole, rabeprazole, esomeprazole, pantoprazole, pariprazole, leminoprazole, SCH 28080, and enantiomers, isomers, free bases, salts, and mixtures of any thereof.

5. The method of claim 1, wherein administering the pharmaceutically acceptable composition comprises inducing apoptosis in the tumor cells.

6. The method of claim 1, further comprising administering the pharmaceutically acceptable composition in a dosage of about 180 mg/day of lansoprazole.

7. The method of claim 1, wherein the tumor cells are selected from the group consisting of: RPM18226, NC37, MC/CAR, SUDHL4, RPMI6666, GDM-1, MOLT3, J45-01, MCF7, HL60 clone 15, P116, SW620, MV-4-11, SKMEL5, DAUDI, DOHH2, HUT102, CCRF-CEM, HUT78, A3, MDA-MB-435, MDA-MB-231, RS4.11, ES-2, IGROV 1, OVCAR5, OVCAR8, J-gamma-1, KU812, NK92MI, 786-O, A498, H522, SNB19, OVCAR4, H9, HH, EKVX, OVCAR5, UACC257, H226, UO-31, NAMALWA, SKMEL28, SKMEL2, M14, H322M, HCC2998, HL60, HT29, A549, RXF393, PC3, H460, MC116, MOLT4, JMI, HOP-62, HCT-15, SF-539, SF295, ST486, U251, and UACC-62.

8. The method of claim 1, wherein the tumor cells are associated with a disease selected from the group consisting of: carcinoma, lymphoma, blastoma, myeloma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, and hepatic carcinoma.

9. The method of claim 1, wherein administering the pharmaceutically acceptable composition comprises administering the proton pump inhibitor in an amount of about 10 mg/kg to about 100 mg/kg.

10. The method of claim 1, further comprising administering a second agent wherein the second agent is a chemotherapeutic agent.

11. The method of claim 1, further comprising administering a buffering agent.

12. A method of killing a growth deregulated cell, the method comprising:
administering to a subject an effective amount of a pharmaceutical composition comprising lanzoprazole in an amount of about 120 mg to about 300 mg, wherein upon administration to the subject the composition interacts with a mass of growth deregulated cells outside of the subject's gastric lumen; wherein the lansoprazole induces apoptosis in the growth deregulated cells; and wherein a mass of growth deregulated cells is reduced in size after about three weeks from the administration.

13. The method of claim 12, wherein upon the administration of the pharmaceutically composition the survival rate of a subject is greater than about 15% as compared to a subject administered a placebo.

14. The method of claim 2, further comprising determining that the subject is not suffering from elevated gastric acid production.

15. (canceled)

16. (canceled)

17. A pharmaceutical composition for use in reducing the size of a tumor in a subject, wherein the pharmaceutical composition comprises:
a proton pump inhibitor or pharmaceutically acceptable salt thereof, in an amount to treat the tumor in the subject;
a pharmaceutically acceptable excipient; and
instructions for administering the proton pump inhibitor to the subject suffering from the tumor so as to treat the tumor.

18. The method of claim 1, wherein administering the pharmaceutically acceptable composition comprises administering from about 20 mg to about 400 mg of lansoprazole.

19. The method of claim 18, wherein administering the pharmaceutically acceptable composition including lansoprazole further comprises lowering the pH of the tumor cells.

20. The method of claim 1, wherein administering a pharmaceutically acceptable composition comprising an effective amount of a proton pump inhibitor comprises administering a pharmaceutically acceptable composition comprising an effective amount of a substituted benzimidazole compound having H⁺/K⁺ ATPase inhibiting activity.

21. The method of claim 1, wherein administering the pharmaceutically acceptable composition comprises inducing apoptosis in the tumor cells by modifying the K⁺ level of the tumor cells.

22. The method of claim 1, wherein the tumor cells are hepatoma cells.

23. A method of treating cancer, comprising:
administering to a patient in need thereof a pharmaceutically effective amount of lansoprazole or a pharmaceutically acceptable salt thereof; and
inhibiting growth of a tumor.

24. The method of claim 23, wherein the inhibition of growth is measured as a delay in tumor doubling time.

25. The method of claim 24, wherein the tumor doubling time is extended by a factor of at least two.

26. The method of claim 23, wherein the volume of the tumor is reduced by at least 10%.

27. The method of claim 23, wherein the patient has a cancerous tumor.

28. The method of claim 23, wherein the cancer is selected from the group consisting of: carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, and hepatic carcinoma.

29. The method of claim 23, wherein the lansoprazole is administered at a dosage of about 120 mg/day to about 300 mg/day.

30. The method of claim 23, wherein the lansoprazole is administered at a dosage of about 10 mg/kg/day to about 150 mg/kg/day.

31. The method of claim 29, wherein the survival rate of the patient is greater than about 15% as compared to a patient administered a placebo.

32. The method of claim 10, wherein the proton pump inhibitor and the second agent are administered together in the same dosage form.

33. The method of claim 10, wherein the proton pump inhibitor is administered simultaneously with the second agent.
